(19) United States
(12) Patent Application Publication
(10) Pub. No.: US 2018/0168500 A1
(43) Pub. Date: Jun. 21, 2018

(54) System and Method for Identification of Brain Injury

(71) Applicant: MotoMetrix Inc., Omaha, NE (US)

(72) Inventor: Preston Badeer, Omaha, NE (US)

(21) Appl. No.: 15/667,587

(22) Filed: Aug. 2, 2017

Related U.S. Application Data
(60) Provisional application No. 62/370,139, filed on Aug. 2, 2016.

(51) Int. Cl. A61B 5/00; A61B 5/103

(52) U.S. Cl. CPC A61B 5/4064; A61B 5/1036; A61B 5/4023; A61B 2090/064; A61B 5/7282; A61B 5/0002; A61B 5/7278

(57) ABSTRACT

A method for identifying brain injuries in a test subject includes acquiring, at a selected number of instances across a selected time interval, a set of signals indicative of force from a set of force sensors when the test subject stands on a pressure measurement unit. The method includes calculating a center-of-pressure of the test subject at the selected number of instances across the selected time interval based on the acquired set of signals from the set of force sensors. The method includes calculating a metric indicative of randomness of the center-of-pressure of the test subject across the selected time interval. The method includes determining a state of brain health of the test subject based on the metric indicative of randomness of the center-of-pressure. The method includes reporting the state of brain health of the test subject to a user interface.

### CROSS-REFERENCE TO RELATED APPLICATION

The present application claims the benefit under 35 U.S.C. § 119(e) of U.S. Provisional Application Ser. No. 62/370,139, filed Aug. 2, 2016, entitled AUTOMATIC IDENTIFICATION OF DISEASE AND TRAUMA, naming Preston Badeer as an inventor, which is incorporated herein by reference in the entirety.

### TECHNICAL FIELD

The disclosure generally relates to the field of identifying brain injuries and brain disease, and, more particularly, to the automatic detection of brain injuries and brain disease based on changes in physiological characteristics of an individual.

### BACKGROUND

Existing field tests to identify brain injuries usually include a simple questionnaire. These questionnaires are typically administered by non-health care professionals. For example, questionnaires are commonly administered to players by coaches on the sideline of a football field. One drawback of this current method is that those individuals administering the questionnaires usually are not properly trained to identify brain injuries. Another drawback of the current system is that the questionnaire often must be conducted shortly after the brain injury has occurred. Furthermore, analyzing questionnaire results is highly subjective, and the administrator may have no reliable standard with which to compare the results of the questionnaire. Still further, state of the art systems for detecting brain injuries are time consuming, labor intensive, and require equipment unsuitable for field tests. Therefore, there exists a need for a system and method, which cure one or more of the shortcomings identified above.

### SUMMARY

A system for identifying brain injury in a test subject is disclosed, in accordance with one or more embodiments of the present disclosure. In one embodiment, the system includes a pressure measurement unit. In another embodiment, the pressure measurement unit includes a set of force sensors. Each force sensor of the set of force sensors may be configured to output a signal indicative of a force applied to the force sensor. In another embodiment, the system includes a memory. In another embodiment, the system includes one or more processors communicatively coupled to each of the force sensors. In another embodiment, the one or more processors are configured to execute a set of program instructions which cause the processor to receive a set of signals, acquired at a selected number of instances across a selective time interval, indicative of force from at least some of the set of force sensors when a test subject stands on the pressure measurement unit; calculate a center-of-pressure of the test subject at the selected number of instances across the selected time interval based on the received set of signals from the set of force sensors; calculate a metric indicative of randomness of the center-of-pressure across the selected time interval; determine a state of brain health of the test subject based on the metric indicative of randomness of the center-of-pressure; and/or report the state of brain health of the test subject to a user interface.

A system for identifying brain injury in a test subject is disclosed, in accordance with one or more additional embodiments of the present disclosure. In one embodiment, the system includes a pressure measurement unit. In another embodiment, the pressure measurement unit includes a set of force sensors. Each force sensor of the set of force sensors may be configured to output a signal indicative of a force applied to the force sensor. In another embodiment, the system includes a memory. In another embodiment, the system includes one or more processors communicatively coupled to each of the force sensors. In another embodiment, the one or more processors are configured to execute a set of program instructions which cause the processor to receive a set of signals, acquired at a selected number of instances across a selective time interval, indicative of force from at least some of the set of force sensors when a test subject stands on the pressure measurement unit; calculate a center-of-pressure of the test subject at the selected number of instances across the selected time interval based on the received set of signals from the set of force sensors; calculate a variability of the center-of-pressure across the selected time interval; determine a state of brain health of the test subject based on the variability of the center-of-pressure; and/or report the state of brain health of the test subject to a user interface.

A system for identifying brain injury in a test subject is disclosed, in accordance with one or more additional embodiments of the present disclosure. In one embodiment, the system includes a pressure measurement unit. In another embodiment, the pressure measurement unit includes a set of force sensors. Each force sensor of the set of force sensors may be configured to output a signal indicative of a force applied to the force sensor. In another embodiment, the system includes a memory. In another embodiment, the system includes one or more processors communicatively coupled to each of the force sensors. In another embodiment, the one or more processors are configured to execute a set of program instructions which cause the processor to receive a set of signals, acquired at a selected number of instances across a selective time interval, indicative of force from at least some of the set of force sensors when a test subject stands on the pressure measurement unit; calculate a metric indicative of randomness of a center-of-pressure across the selected time interval; calculate a variability of the center-of-pressure across the selected time interval; determine a state of brain health of the test subject based on the metric indicative of randomness of the center-of-pressure; and/or report the state of brain health of the test subject to a user interface.

A method for identifying brain injury in a test subject is disclosed, in accordance with one or more additional embodiments of the present disclosure. In one embodiment, the method includes acquiring, at a selected number of instances across a selected time interval, a plurality of signals indicative of force from a plurality of force sensors when the test subject stands on a pressure measurement unit. In another embodiment, the method includes calculating a center-of-pressure of the test subject at the selected number of instances across the selected time interval based on the acquired set of signals from the plurality of force sensors. In another embodiment, the method includes calculating a metric indicative of randomness of the center-of-pressure of the test subject across the selected time interval. In another embodiment, the method includes determining a state of brain health of the test subject based on the metric indicative of randomness of the center-of-pressure, and reporting the state of brain health of the test subject to a user interface.

A method for identifying brain injury in a test subject is disclosed, in accordance with one or more additional embodiments of the present disclosure. In one embodiment, the method includes acquiring, at a selected number of instances across a selected time interval, a plurality of signals indicative of force from a plurality of force sensors when the test subject stands on a pressure measurement unit. In another embodiment, the method includes calculating a center-of-pressure of the test subject at the selected number of instances across the selected time interval based on the acquired set of signals from the plurality of force sensors. In another embodiment, the method includes calculating a variability of the center-of-pressure of the test subject across the selected time interval. In another embodiment, the method includes determining a state of brain health of the test subject based on the variability of the center-of-pressure, and reporting the state of brain health of the test subject to a user interface.

A method for identifying brain injury in a test subject is disclosed, in accordance with one or more additional embodiments of the present disclosure. In one embodiment, the method includes acquiring, at a selected number of instances across a selected time interval, a plurality of signals indicative of force from a plurality of force sensors when the test subject stands on a pressure measurement unit. In another embodiment, the method includes calculating a variability of a center-of-pressure of the test subject across the selected time interval. In another embodiment, the method includes calculating a metric indicative of randomness of the center-of-pressure of the test subject across the selected time interval. In another embodiment, the method includes determining a state of brain health of the test subject based on the variability of the center-of-pressure, and reporting the state of brain health of the test subject to a user interface.

It is to be understood that both the foregoing general description and the following detailed description are exemplary and explanatory only and should not restrict the scope of the claims. The accompanying drawings, which are incorporated in and constitute a part of the specification, illustrate exemplary embodiments of the inventive concepts disclosed herein and together with the general description, serve to explain the principles.

### BRIEF DESCRIPTION OF THE DRAWINGS

The numerous advantages of the embodiments of the inventive concepts disclosed herein may be better understood by those skilled in the art by reference to the accompanying figures in which:

FIG. 1A illustrates a simplified schematic diagram of a system for identifying brain injuries, in accordance with one or more illustrative embodiments of the present disclosure.

FIG. 1B illustrates a simplified schematic diagram of a system for identifying brain injuries, in accordance with one or more illustrative embodiments of the present disclosure.

FIG. 2A illustrates a top view of a pressure measurement unit, in accordance with one or more illustrative embodiments of the present disclosure.

FIG. 2B illustrates a top view of a pressure measurement unit, in accordance with one or more illustrative embodiments of the present disclosure.

FIG. 2C illustrates a top view of a pressure measurement unit, in accordance with one or more illustrative embodiments of the present disclosure.

FIG. 3 illustrates a flowchart of a method for identifying brain injuries, in accordance with one or more illustrative embodiments of the present disclosure.

FIG. 4 illustrates a flowchart of a method for identifying brain injuries, in accordance with one or more illustrative embodiments of the present disclosure.

FIG. 5 illustrates a flowchart of a method for identifying brain injuries, in accordance with one or more illustrative embodiments of the present disclosure.

### DETAILED DESCRIPTION OF THE INVENTION

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and, together with the general description, serve to explain the principles of the invention.

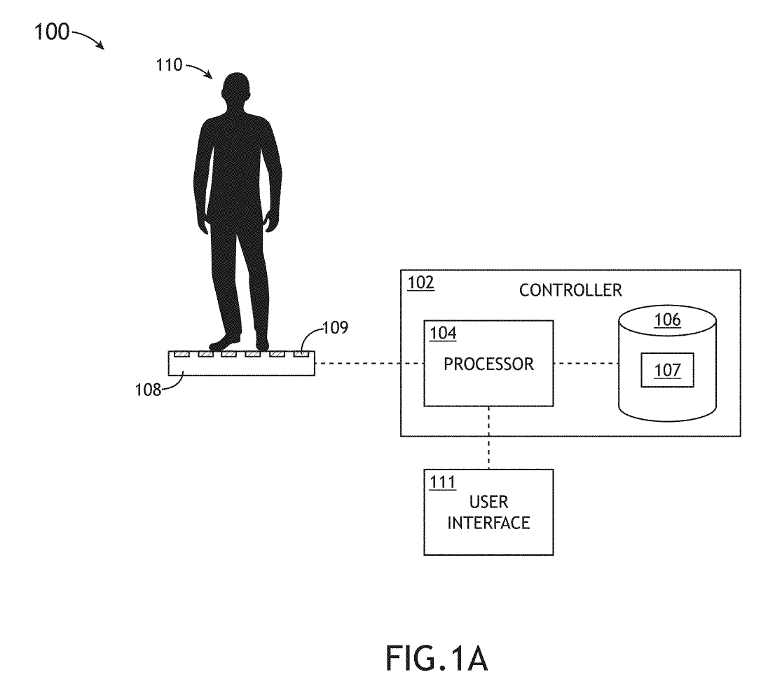

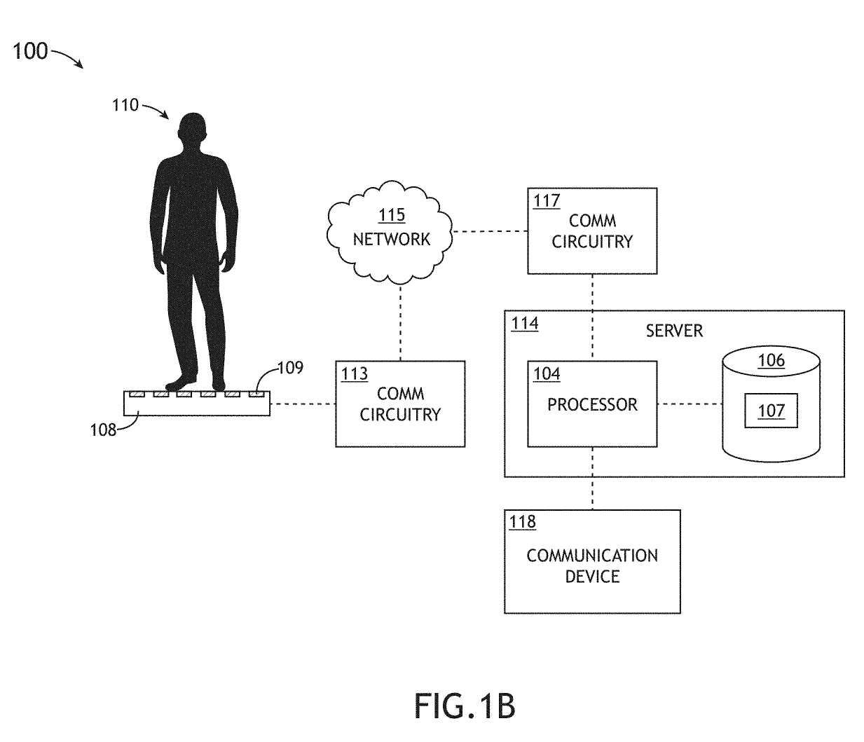

Referring generally to FIGS. 1A through 5, a system and method for identifying brain injuries is described, in accordance with one or more embodiments of the present disclosure.

The systems and methods described herein may be more fully understand in light of: Christian Poellabauer et al., Challenges in Concussion Detection Using Vocal Acoustic Biomarkers, IEEE Access (2015); Ross A. Clark et al., Validity and Reliability of the Nintendo Wii Balance Board for Assessment of Standing Balance, Gait & Posture (2009); Mitsuhiro Hayashibe et al., Subject-Specific Center of Mass Estimation for In-home Rehabilitation-Kinect-Wii board vs. Vicon-Force plate, International Conference of NeuroRehabilitation (2012); Ross A. Clark et al., Instrumenting Gait Assessment Using the Kinect in People Living With Stroke: Reliability and Association With Balance Tests, Journal of NeuroEngineering and Rehabilitation (2015); Shmuel Springer et al., Validity of the Kinect for Gait Assessment: A Focused Review, Sensors (2016); Aaron Staranowicz et al., Evaluating the Accuracy of a Mobile Kinect-based Gait-monitoring Systems for Fall Prediction, University of Texas Arlington (2013); Harrison L. Bartlett et al., Accuracy of Force and Center of Pressure Measures of the Wii Balance Board, US National Library of Medicine (2014); and Moshe Gabel et al., Full Body Gait Analysis with Kinect, Microsoft Research (2016), all of which are hereby incorporated herein by reference in the entirety.

Embodiments of the present disclosure are directed to a system and method for identifying brain illness or traumatic brain injury in a test subject based on physiologic changes in the test subject. Such physiological changes include changes in a person's ability to balance, speech pattern changes, and changes to a person's gait and/or posture. In one embodiment, a test subject may stand on top of a pressure measurement unit including a set of force sensors. As a test subject stands on a pressure measurement unit, the test subject may naturally and slightly sway to maintain balance. As the test subject sways, the pressure measurement unit may determine how the test subject's center-of-pressure (COP) changes throughout the selected time interval. Using signals from force sensors within the pressure measurement unit, one or more processors may determine the test subject's COP at a number of instances across a selected time interval. Then, the one or more processors may calculate one or more metrics indicative of the randomness (e.g., approximate entropy) of the test subject's COP. It is noted that the terms “metric indicative of randomness” and “randomness metric” may be used interchangeably for the purpose of the present disclosure. In another embodiment, the system may be configured to calculate a variability (e.g., difference between high and low value in given time interval) of the test subject's COP. In another embodiment, the system may detect a brain injury or the onset of brain disease based on changes in a test subject's 110 randomness and/or variability of their COP measurements.

In order to quantify randomness of a test subject's COP, some embodiments of the present disclosure are directed to calculating one or more metrics indicative of randomness of the COP measurements. For example, as a test subject stands and naturally sways, their swaying movements may be smooth and random, or they may be more sudden and deliberate (e.g., less random). In one embodiment, a metric indicative of randomness may include approximate entropy. It is noted that herein that higher approximate entropy scores may indicate increased randomness (associated with normal/healthy brain), whereas lower approximate entropy scores may indicate decreased randomness (associated with injured/diseased brain). In order to quantify variability of a test subject's COP, some embodiments of the present disclosure are directed to calculating the variability of a test subject's COP measurements. Variability may include a measurement of how often a test subject's COP changes, how smoothly the COP changes, how dramatically the COP changes, and the like.

Once acquired the metrics indicative of randomness and variability of a test subject's COP measurements may be used in order to determine whether the test subject has suffered a brain injury or is experiencing onset of a brain-related disease. As a healthy person stands, they tend to naturally and sway to maintain balance. This natural swaying, while slight, is done in a substantially smooth and random manner. As such, a COP measurements from healthy test subject are constantly and smoothly changing. In comparison, as a person with a brain injury stands, they tend to stand more rigidly without swaying. This less-organic stance may be due to the fact that the brain injury has caused a decrease in their ability to balance. Instead of naturally swaying, a person with a brain injury may tend to lock their stance to one side, then suddenly and deliberately “jerk” their stance in order to maintain balance. As such, the COP measurements of a test subject with a brain injury tend to remain relatively constant, then suddenly change. This causes the metrics indicative of randomness and/or the variability of a brain-injured test subject's COP measurements to be low relative to that of a healthy test subject.

In this regard, it may be the case that a healthy test subject 110 may exhibit higher variability and randomness metrics scores, whereas a test subject 110 with a brain injury or brain disease may exhibit lower variability and randomness metrics scores. Further, in cases where a test subject is consistently monitored, a decrease in randomness and/or variability over time may be an indication of a recent brain injury or onset of brain disease.

FIG. 1A illustrates a simplified block diagram of a system 100 for identifying brain injuries, in accordance with one or more embodiments of the present disclosure. In one embodiment, system 100 may include a pressure measurement unit 108 and a controller 102. In another embodiment, the pressure measurement unit 108 includes a set of force sensors 109. The controller 102 may include one or more processors 104 and memory 106.

In one embodiment, pressure measurement unit 108 may be communicatively coupled to thee one or more processors 104 of the controller 102. For example, each force sensor 109 from the set of force sensors 109 may be communicatively coupled to the one or more processors 104 of the controller 102. In another embodiment, force sensors 109 are configured to transmit force measurements to controller 102. In another embodiment, force measurements are stored in a database 107 within memory 106. In another embodiment, each force measurement is time stamped. For example, as a test subject 100 stands on the force measurement unit 108, each of the force sensors 109 may transmit force measurements to controller 102. As the controller 102 receives each force measurement, the force measurements from each of the force sensor 109 may be time stamped and stored in memory 106.

In another embodiment, the one or more processors 104 are configured to execute a set of program instructions stored in memory 106 to cause the one or more processors 104 to carry out one or more steps of the present disclosure. In one embodiment, the one or more processors 104 are configured to receive a set of signals, acquired at a selected number of instances across a selected time interval, indicative of force from at least some of the set of force sensors when a test subject 110 stands on the pressure measurement unit. In another embodiment, the one or more processors 104 are configured to calculate a center-of-pressure of the test subject 110 at the selected number of instances across the selected time interval based on the received set of signals from the at least some of the set of force sensors. In another embodiment, the one or more processors 104 are configured to calculate a metric indicative of randomness of the center-of-pressure of the test subject 110 across the selected time interval. In another embodiment, the one or more processors 104 are configured to calculate a metric indicative of randomness of the center-of-pressure of the test subject 110 across the selected time interval. In another embodiment, the one or more processors 104 are configured to determine a state of brain health of the test subject 110 based on the metric indicative of randomness of the center-of-pressure across the selected time interval. In another embodiment, the one or more processors 104 are configured to report the state of brain health of the test subject 110 to a user interface.

In another embodiment, the pressure measurement unit 108 may be configured to detect the pressure exerted by a test subject 110 over a selected time interval that the test subject 110 stands on the pressure measurement unit 108. In one embodiment, each force sensor 109 may measure the force/pressure exerted on it at a set of samplings/instances across a selected time interval. It is noted that a selected time interval may have any length and corresponds to the time that the test subject is standing on the pressure measurement unit 108. For example, a selected time interval may have a length between 1 s and 10 mins. In addition, each selected time interval may include any number of sampling/measurement instances. In this regard, the sampling/measurement instance represents the instances with which force/pressure data is acquired from the force sensors 109 during a particular measurement interval. For example, the sampling frequency/measurement instances may occur at selected times within a given measurement interval that a test subject 110 is standing on a pressure measurement unit 108. For instance, a sampling/measurement instance may occur at any time frame less than the selected time interval. The sampling/measurement instance may occur at a rate between 0.01 s and 5 mins. For example, a test subject 110 may stand on a pressure measurement unit 108 for a minute-long selected time interval. During that minute time interval, each of the force sensors 109 may measure the force/pressure exerted on it multiple times throughout that minute. For example, each of the force sensors 109 may measure the force/pressure apply to it by the test subject 108 every 1 second of the 1 minute selected time interval.

It is noted that the timing information providing above for the selected time intervals and measurement instances/sampling frequencies are provided merely for illustrative purposes and do not represent a limitation on the scope of the present disclosure.

It is recognized herein that a test subject's 100 center-of-pressure may be related to multiple values/mechanisms including, but not limited to, a test subject's 110 center-of-mass. For example, as a test subject 110 stands on the pressure measurement unit 108, they may naturally slightly sway from left to right, from their heels to their toes, and the like. As the test subject 110 naturally sways, each force sensor 109 of the set of force sensors 109 may detect varying force measurements, and transmit these measurements to the controller 102. As the processor 104 receives each time-stamped set of force measurements, it may calculate a different center-of-pressure for each set of measurements, and time stamp each center-of-pressure measurement. In another embodiment, center-of-pressure measurements may be stored in database 107 of memory 106 and used for future comparative analysis.

In one embodiment, the set of program instructions may cause the processor 104 to calculate a test subject's 110 center-of-pressure (COP) at a number of instances across a selected time interval. For example, processor 104 may calculate a test subject's 110 COP for each set of force measurements received at each instance within the selected time interval. For instance, carrying on with the example above, if processor 104 was configured to receive fifty sets of force measurements (e.g., fifty instances) throughout a selected time interval that a test subject 110 stands on a pressure measurement unit 108, processor 104 may also be configured to calculate fifty COP measurements.

In one embodiment, processors 104 may calculate a randomness metric of a test subject's 110 COP measurements using one or more algorithms. In one embodiment, the randomness metric may be a function of, but not limited to, how randomly a test subject 110 sways as they stand on the pressure measurement unit 108, how often the COP changes as a test subject 110 stands on the pressure measurement unit 108 (e.g., COP change frequency), the extent to which the COP changes (e.g., COP range), how fast the COP changes (e.g., COP change rate), and the like. In one embodiment, a randomness metric may be measured through the calculation of the approximate entropy associated with the acquired COP measurements. In this regard, the randomness metric may include the approximate entropy. Approximate entropy used in the context of medical analysis is described generally in Pincus, S. M.; Gladstone, I. M.; Ehrenkranz, R. A. (1991). “A REGULARITY STATISTIC FOR MEDICAL DATA ANALYSIS”. Journal of Clinical Monitoring and Computing. 7 (4): 335-345, which is incorporated herein by reference. Approximate entropy is also described in “Approximate entropy as a measure of system complexity”. Proceedings of the National Academy of Sciences. 88 (6): 2297-2301, which is incorporated herein by reference in the entirety.

It is noted that the approximate entropy of a test subject's 110 COP may be indicative of their overall brain health. In one embodiment, approximate entropy may be rated on a unit-less scale from 0.0 to 2.0. In another embodiment, high approximate entropy scores may indicate good overall brain health, whereas low approximate entropy scores may indicate potential brain trauma. For example, a healthy test subject 110 with no brain damage may naturally and slightly sway as they stand on the pressure measurement unit 108. As the test subject 110 naturally sways, their COP may change frequently, which results in high approximate entropy. On the other hand, a test subject 110 with a brain irregularity (e.g., a concussion, a brain tumor, a brain disease, and the like) may tend to stand more rigidly. The brain damage may cause the test subject 110 not to slightly sway as a healthy test subject 110 may, but rather stand in a more rigid and robotic manner. In this regard, it may be the case that a healthy test subject 110 may exhibit a higher approximate entropy score, whereas a test subject 110 with brain damage may exhibit a lower approximate entropy score. While an absolute indicator of brain irregularity is challenging, it has been found that changes in approximate entropy over time for a monitored individual provide a quality indicator of changes in brain health.

Brain injuries may cause approximate entropy scores to vary due to many factors. These factors may include, but are not limited to, a decreased ability to balance. A brain injury may thus result in a decreased ability to balance, and cause the test subject 110 to exhibit a less organic stance. As opposed to test subjects 110 with healthy brains who may naturally and smoothly sway to adjust their balance, test subjects 110 with brain injuries may tend to lean to one side, jerk to adjust balance, suddenly shift their weight, and the like. As a result, the movements of test subject 110 with brain injuries are less random, resulting in reduced randomness and lower approximate entropy scores. In comparison, test subjects 110 with no brain trauma naturally and smoothly sway in a substantially random manner, resulting in a higher randomness score and higher approximate entropy scores. In this respect, the system 100 may relate high approximate entropy scores to users with a healthy brain, and low approximate entropy scores to users having experienced brain trauma or another brain irregularity.

In another embodiment, the one or more processors 104 may be configured to calculate the variability of a test subject's 110 COP measurements. In one embodiment, variability scores may be calculated in addition to randomness metrics. In one embodiment, variability may be dependent on how often the COP changes as a test subject 110 stands on the pressure measurement unit 108 (e.g., COP change frequency), the extent to which the COP changes (e.g., COP range), how fast the COP changes (e.g., COP change rate), and the like. For example, as a test subject 110 with no brain trauma stands on the pressure measurement unit 108, they may naturally sway to maintain balance. This may result in a high variability score. By way of another example, as a test subject 110 a brain injury stands on the pressure measurement unit 108, they may stand more rigidly as a result of their decreased ability to balance. This may result in a lower variability score. In this regard, high variability scores may be indicative of a healthy brain, whereas low variability scores may be indicative of brain trauma.

In embodiments where the one or more processors 104 calculate both randomness metrics and variability of a test subject's 110 COP, the one or more processors 104 may be configured to calculate an overall brain analysis score. For example, an overall brain analysis score may be rated on a numerical scale. For instance, overall brain analysis scores may be rated on a scale from 1 to 10, wherein 1 indicates a low probability of brain trauma, and a 10 indicates a high probability for brain trauma. It is noted that other scales may be implemented without departing from the spirit and scope of the present disclosure.

In another embodiment, the set of program instructions may be configured to cause the processor 104 to determine a state of brain health. As previously noted, a test subject's 110 overall brain health may be a function of, but not limited to, randomness metrics (e.g., approximate entropy, and the like), variability, and the like. Alternatively, processor 104 may determine overall brain health as a combination of multiple factors. For example, processor 104 may determine overall brain health as a function of both randomness variables (e.g., approximate entropy, and the like) and variability.

In another embodiment, the set of program instructions may be configured to cause the processor 104 to report a test subject's 110 overall brain health state. In one embodiment, reporting a test subject's 110 brain health may include displaying the report on the user interface 111. In an alternative embodiment, the one or more processors 104 may report a test subject's 110 brain health by delivering a message to a user's (e.g., a test subject 110, a test subject's 110 doctor, a test subject's 110 family, and the like) computer, tablet, smartphone, and the like. It is contemplated that the system 100 may use any method of message delivery known in the art including, but not limited to, emails, text messages, instant messages, application notifications, and the like. In one embodiment, reporting a test subject's 110 overall brain health state may include reporting a calculated brain health score. In one embodiment, reporting a test subject's 110 overall brain health state may include reporting information regarding a healthy brain or the possibility of a brain injury. For example, reporting overall brain health may include, but is not limited to, reporting healthy brain function, reporting deteriorated brain function, reporting one or more brain injuries, reporting one or more brain injuries, reporting one or more brain diseases, and the like.

It is noted that reports regarding a test subject's 110 brain health state may provide users with information regarding, but not limited to, brain trauma, brain diseases, risk of brain diseases, potential brain injuries, and the like. Furthermore, system 100 may be used to determine the severity of a brain injury, likelihood of a brain injury, rate of rehabilitation, speed of recovery, level of recovery, and the like. System 100 may also be used as an early warning system for brain trauma. It is further noted that, as overall brain health scores are calculated over time, that ranges of brain health scores may be associated with different types and/or severities of brain injuries. It is further contemplated that, because a test subject's 110 COP measurements, variability scores, randomness metric scores (e.g., approximate entropy scores, and the like), and overall brain health scores may be stored in database 107 of memory 106, additional algorithms may be developed in the future to retroactively analyze a test subject's 110 measurements. It is noted that the system 100 may provide valuable information to doctors and medical professionals who may be studying a particular test subject 110, groups of test subjects 110, or brain damage in general.

In at least one embodiment, certain features of a test subject's 110 measurements and scores may be indicative of brain injury. Brain injuries that may be detectable via changes in a person's ability to balance include traumatic brain injury, second impact syndrome, recurrent traumatic brain injury, closed brain injury, diffuse axonal injury, concussions, and the like. For example, a test subject's 110 measurements may be graphed. In this regard, first or second derivatives of the graph may have certain absolute quantities corresponding to deteriorated gross motor controls.

In another embodiment, system 100 may be used to track a person's balance and brain health over a period of time. For example, a test subject 110 may stand on the pressure measurement unit 108 for selected time intervals over a period of days, weeks, or months. In another embodiment, processor 104 may be configured to run a set of program instructions stored in memory 106 configured to determine one or more of COP measurements, variation scores, randomness metric scores, and overall brain health scores for each selected time interval the test subject 110 stands on the pressure measurement unit 108. In this manner, the system 100 may track a test subject's 110 overall brain health over time.

Furthermore, in another embodiment, by tracking a test subject's 110 overall brain health over time, the system 100 may be able to develop a composite “baseline” score for each test subject 110. For example, a test subject 110 may end each workout over a period of several months by standing on the pressure measurement unit 108 for a selected time interval. For each selected time interval the person 108 stands on the pressure measurement unit 108, the test subject's 110 COP measurements, variation scores, approximate entropy scores, and overall brain health scores may be determined and stored in database 107 of memory 106. After each new center point entry, system 100 may average each measurement to obtain a continuing baseline score.

In another embodiment, a test subject's 110 measurements may be compared to the test subject's previous healthy “baseline” scores. It is noted herein that determining a healthy baseline score may allow the present disclosure to provide more accurate, objective results and to ameliorate erroneous results. For example, processors 104 may compare a test subject's 110 later scores to that of the test subject's earlier, known, healthy baseline score in order to identify a relative change in the test subject's 110 ability to maintain balance. Relative changes may indicate that the test subject 110 has suffered from a concussion or other brain injury. The processor 104 may then report the brain injury via user interface 111.

In one embodiment, reporting a test subject's 110 measurements and scores may include displaying a comparison between the test subject's 110 current score and their previous healthy baseline score. In other embodiments, comparisons may include direct comparisons (e.g., differences in amplitude, frequency, deviation from a centerline, and the like), comparisons of derived data (e.g., smoothness of balance transitions, and the like), comparisons to the scores of other test subjects 110 with known brain injuries, and the like.

In another embodiment, baseline scores of a plurality of test subjects 110 in a particular community may be aggregated to produce a communal baseline score for comparison during testing. It is noted that communal baseline scores may be created for particular demographics. For example, one baseline balance score may include data from high school football players. By way of another example, a baseline balance score may include data from a single football team. By way of another example, a baseline balance score may include data from individuals over 65. By way of another example, a baseline score may include data from female soccer players between the ages of 9 and 12.

It is noted that by creating baseline scores for a wide variety of demographics, the present disclosure may be used to more accurately identify brain injuries in individuals who have not used the present disclosure to create their own personalized baseline score. For example, a high school football player who has never used system 100 before and who does not have his own baseline score may nonetheless make use of the present disclosure by using system 100 and comparing his COP measurements, variation scores, approximate entropy scores, and overall brain health scores to those of an aggregated baseline score for a demographic group including multiple high school football players. It is noted that test subjects 110 may use system 100 to compare their COP measurements, variation scores, approximate entropy scores, and overall brain health scores to those of a demographic having any one or more characteristics in common with the test subject 110. Characteristics which may be considered in developing different demographics and baseline scores may include, but are not limited to, age, height, weight, gender, individuals with similar brain health histories, and the like.

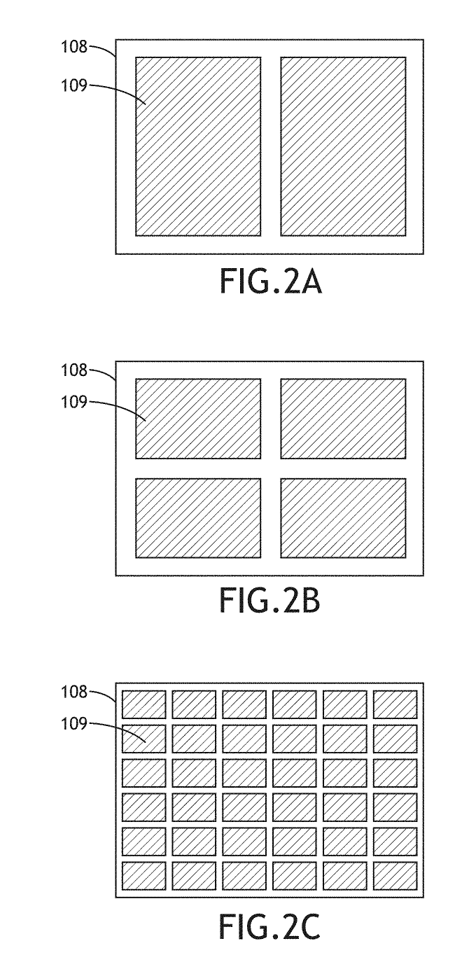

FIGS. 2A-2C illustrate top views of various force sensor layout configurations of the pressure measurement unit 108 in accordance with one or more embodiments of the present disclosure. As depicted in FIGS. 2A-2C, pressure measurement unit 108 may include a set of force sensors 109 arranged in an M×N grid. In one embodiment, as shown in FIG. 2A, pressure measurement unit 108 may include a set of force sensors 109 arranged in a 2×1 grid. By way of another example, FIG. 2B pressure measurement unit 108 may include a set of force sensors 109 arranged in a 2×2 grid. By way of another example, pressure measurement unit 108 may include a set of force sensors 109 arranged in a 6×6 grid.

It is noted herein that the examples depicted in FIGS. 2A-2C are provided merely for illustrative purposes and should not be interpreted to limit the scope of the present disclosure. It is further noted that the pressure measurement unit 108 may include any arrangement of force sensors 109, including, but not limited to, any M×N array of force sensors 109, an offset-array of force sensors 109, a non-array arrangement of force sensors 109, and the like.

The force sensors 109 may include any type of force/pressure sensing device known in the art. For example, the force sensors 109 may include, but are not limited to, one or more tension links, one or more force plates, one or more load pins, one or more load cells, one or more piezoelectric sensors, one or more force sensitive resistors (FSRs), one or more force sensitive capacitors (FSCs), and the like. It is noted that force sensors 109 may include any shape of force sensor 109 including, but not limited to, a square, a rectangle, a hexagon, a circle, an ellipse, and the like.

It is noted herein that the use of numerous, smaller sensors may allow a pressure measurement unit 108 to more accurately measure the force exerted by a test subject 110, thereby allow a mapping of the pressure across the contact surface between the test subject and the surface of the pressure measurement unit 108. For example, as a test subject 110 stands on the pressure measurement unit 108 depicted in FIG. 2A, each of the test subject's 100 feet may be placed on a single grid of the 2×1 grid of force sensors 109. In this example, the pressure measurement unit 108 may only be able to detect changes in pressure from left to right, and vice versa. By way of another example, as a test subject 110 stands on the pressure measurement unit 108 depicted in FIG. 2C, each of the test subject's 100 feet may be placed on several sensors of the 6×6 grid of force sensors 109. In this example, the pressure measurement unit 108 may be able to more accurately determine the force and/or pressure exerted on the pressure measurement unit 108 as a function of position, including pressure changes from left to right, as well as pressure changes from front to back (e.g., heel to toe, and vice versa). It is noted that varying numbers of force sensors 109 may allow embodiments of the present disclosure to be more finely tuned to a user's needs, including, but not limited to, sensitivity, accuracy, price, size, and the like.

The pressure measurement unit 108 may be embodied in any form known in the art. For example, the pressure measurement unit 108 may be a force plate or a set of force plates. By way of another example, the pressure measurement unit 108 may be a digital scale or a set of digital scales.

While much of the present disclosure focuses on the pressure measurement unit 108 in the context of a dedicated measurement device, it is contemplated herein that the present disclosure may extend to additional structural configurations. In one embodiment, the pressure measurement unit 108 may be embodied as a pair of shoes, whereby each shoe includes one or more force sensors 109 embedded in the sole of the shoe.

The one or more processors 104 of controller 102 may include any one or more processing elements known in the art. In this sense, the one or more processors 104 may include any microprocessor-type device configured to execute software algorithms and/or instructions. In one embodiment, the one or more processors 104 may consist of a desktop computer, mainframe computer system, workstation, image computer, parallel processor, or other computer system (e.g., networked computer) configured to execute a program configured to operate the system 100, as described throughout the present disclosure. It should be recognized that the steps described throughout the present disclosure may be carried out by a single computer system or, alternatively, multiple computer systems. In general, the term “processor” may be broadly defined to encompass any device having one or more processing elements, which execute program instructions from memory 106. Moreover, different subsystems of the system 100 (e.g., pressure measurement unit 108) may include processor or logic elements suitable for carrying out at least a portion of the steps described throughout the present disclosure. Therefore, the above description should not be interpreted as a limitation on the present disclosure but merely an illustration.

The memory 106 may include any storage medium known in the art suitable for storing program instructions executable by the associated one or more processors 104. For example, the memory 106 may include a non-transitory memory medium. For instance, the memory 106 may include, but is not limited to, a read-only memory, a random access memory, a magnetic or optical memory device (e.g., disk), a magnetic tape, a solid state drive and the like. In another embodiment, the memory 106 is configured to store one or more results from the pressure measurement unit 108 and/or the output of the various steps described herein. It is further noted that memory 106 may be housed in a common controller housing with the one or more processors 104. In an alternative embodiment, the memory 106 may be located remotely with respect to the physical location of the one or more processors 104 and controller 102. For instance, the one or more processors 104 of controller 102 may access a remote memory (e.g., server), accessible through a network (e.g., internet, intranet, and the like). In another embodiment, the memory 106 maintains program instructions for causing the one or more processors 104 to carry out the various steps described through the present disclosure.

In one embodiment, a user interface 111 is communicatively coupled to the controller 102. The user interface may include any user input device known in the art. For example, the user input device may include, but is not limited to, a keyboard, a keypad, a touchscreen, a scroll bar, a steering wheel, a joystick, or the like. Those skilled in the art should recognize that a large number of user input devices may be suitable for implementation in the present invention, and that the present invention is not limited to those user input devices listed herein.

In other embodiments, the user interface includes a display used to display data of the system 100 or the pressure measurement unit 108 to a user. The display of the user interface may include any display known in the art. For example, the display may include, but is not limited to, a liquid crystal display (LCD), an organic light-emitting diode (OLED) based display or a CRT display. Those skilled in the art should recognize that any display device capable of integration with a user interface is suitable for implementation in the present disclosure. In another embodiment, a user may user may input selections and/or instructions responsive to data displayed to the user via the user interface.

In another embodiment, center-of-pressure measurements may be presented to a user through a display of the user interface 111. For example, COP measurements may be presented to a user via the user interface 111 in many different forms. For instance, COP measurements may be presented as a simple mark on a two-dimensional (2D) surface, wherein a mark in the center of the surface represents a centered COP, and a mark to the right of center represents a right-leaning COP, and the like. In another embodiment, COP may be displayed in real or near-real time throughout the selected time interval that a test subject 110 stands on the pressure measurement unit 108. Alternatively, in another embodiment, when a COP is to be displayed from data stored in database 107 of memory 106, the test subject's 110 COP may be displayed in near-real time.

Referring now to FIG. 1B, in one embodiment, the one or more processors 104 and memory 106 are embodied in a remote server 114. In one embodiment, system 100 includes communication circuitry 113, 117. In another embodiment, system 100 includes network 115. In this embodiment, pressure measurement unit 108 and force sensors 109 may be configured to transmit force and/or pressure data to the server 114 via communication circuitry 113, 117, and network 115. In this regard, communication circuitry 113, 117 may include any communication circuitry device suitable for interfacing with network 115 known in the art. For example, communication circuitry 113, 117 may include wireline-based interface devices (e.g., DSL-based interconnection, cable-based interconnection, T9-based interconnection, and the like). By way of another example, the communication circuitry devices may include a wireless-based interface device employing GSM, GPRS, CDMA, EV-DO, EDGE, WiMAX, LTE, Wi-Fi protocols, and the like.

It is noted that the discussion above regarding controller 102, processors 104, memory 106, and program instructions with respect to FIG. 1A may be extended to the server 114, processors 104, memory 106, and program instructions of FIG. 1B and is not repeated here for purposes of brevity.

In another embodiment, server 114 may be communicatively coupled to one or more communication device 118. The one or more communication devices 118 may include any communication device capable of interfacing with a server, such as, but not limited to, a computer, a mobile device (e.g., smartphone, tablet, and the like), or a dedicated device. In this regard, upon determining a state of brain health of a test subject 110, the server 114 may report the state of a brain health to the one or more communication devices 118.

In another embodiment, one or more additional devices may be used in conjunction with the pressure measurement unit 108 in order to more accurately identify brain injuries or brain disease. For example, video devices, audio devices, and the like may be used in conjunction with the pressure measurement unit 108 of the present disclosure in order to more accurately identify brain injuries.

In one embodiment, one or more video devices may be utilized in the present system while a test subject 110 is standing on the pressure measurement unit 108. For example, video devices may be used to receive video data of a test subject 110 while they are standing on a pressure measurement unit 108. In this regard, the motion tracking data from the video device may be used in conjunction with the force/pressure measurements. For example, a healthy test subject 110 may be naturally swaying while standing on the pressure measurement unit 108, but may be shifting their weight simultaneously such that their approximate entropy and variability scores are low, indicating potential brain damage. In this example, the use of video data may be used in conjunction with the force/pressure data in order to detect the test subject's 110 natural sway, and help determine that the healthy test subject 110 does not have a brain injury despite low approximate entropy and variability scores.

In another embodiment, video devices may be used to collect video data which may be used to perform a gait analysis. In this regard, a gait analysis may allow system 100 to more accurately identify potential brain injuries. In one embodiment, processors 104 may receive video data from one or more video devices. The video data may include, but is not limited to, depth of field data, RGB data, or any other data useful for producing a 3D motion model. In this regard, the one or more processors 104 may create a skeletal structure of each test subject 110 and derive a 3D model of each skeletal structure moving over time. Processors 104 may then analyze the derived 3D models to identify movements indicative of a potential brain injury.

In another embodiment, one or more video devices may be utilized in the present system in order to actively or passively record test subjects 110 in common postures. For example, one or more video devices may be configured to record test subjects 110 walking, standing, sitting, leaning, and the like. In this regard, processors 104 may process the video data from the video devices to determine a skeletal structure of the test subject 110. In this embodiment, the true posture and positioning of the test subject 110 may be determined. In another embodiment, the simulated skeletal posture data collected from the video devices may be used in conjunction with other data sources (e.g., pressure data, gait analysis data, other video data, and the like) in order to more accurately identify signs of brain trauma, degradation, disease, recovery, improvement, and the like.

In another embodiment, system 100 may include one or more audio devices. In one embodiment, processors 104 receive an audio sample from a test subject 110 via one or more audio devices. The audio sample may be analyzed for certain predetermined speech patterns including, but not limited to, specific biomarkers. The specific biomarkers may be indicative of certain physiological conditions, and may be used to more accurately identify potential brain injuries. Similar to the video devices, audio devices may be used in conjunction with the pressure measurement unit 108 in order to more accurately identify brain injuries.

In some embodiments, a brain health score may be generated by the one or more processors 104 of system 100. The brain health score may represent an aggregation of individual test scores, such as, variability scores, randomness metrics scores, gait and posture analysis scores, audio biomarker scores and the like. The individual scores may be combined and/or weighted in any suitable manner in order to achieve a reliable result. In one embodiment, variability score, gait analysis scores, posture analysis scores, and/or audio biomarker scores may serve as score adjustments to the primary COP randomness metric score.

In embodiments which include at least one or more video devices and one or more audio devices in conjunction with pressure measurement unit 108, the one or more video devices or one or more audio devices may, individually, determine their own risk profile score. In this regard, the risk profile scores from the video devices and/or the audio devices may be factored in to the variability scores and randomness metrics scores from the force/pressure measurements in order to determine an overall brain analysis score. As noted previously, a brain analysis score may be rated on a scale from 1 to 10, wherein 1 indicates a low probability of brain trauma, and a 10 indicates a high probability for brain trauma. It is noted that other scales may be implemented without departing from the spirit and scope of the present disclosure.

In one embodiment, in order to quantify overall brain analysis scores (e.g., scores 1-10) into percentages representing probability of brain injuries, the embodiments of the present disclosure may be configured to compare test subjects' 110 data scores (e.g., variability scores, randomness metrics scores, gait and/or posture analysis scores, audio biomarker scores, and the like) with the diagnoses of health care providers. In other words, embodiments of the present disclosure may compare a test subject's 110 overall brain analysis score with that same test subject's 110 health care provider's diagnosis. In this regard, the health care provider's diagnosis may serve as a “measuring stick” with which to quantify overall brain analysis scores of the present disclosure in to percentages indicating probability of brain injuries.

For example, out of a particular demographic set of test subjects 110, those test subjects 100 with overall brain analysis scores between 7.5 and 8.5 may have been diagnosed with a concussion by a doctor 87% of the time. In this example, the system 100 of the present disclosure may then determine that, individuals in that demographic of test subjects 110 with overall brain scores between 7.5 and 8.5 have an 87% probability of having suffered a concussion. It is noted that other methods of comparing scores from the present disclosure to the health care provider's diagnoses may be implemented without departing from the spirit or scope of the present disclosure.

It is further contemplated herein that one or more machine learning processes may be applied to one or more datasets of the present disclosure to provide a determination of brain health. In one embodiment, measurements taken from test subjects with known brain health states (i.e., known based on clinical diagnosis by health care provider) may be used to generate a classier. For example, COP randomness, COP variability, gate analysis scores, posture analysis scores, biomarker scores and/or demographic data obtained from test subjects with known brain health states may be used to train the classifier. For instance, the controller of system 100 may be used to determine the COP randomness and COP variability of a group of test subjects (e.g., 100-10,000 test subjects) with known demographic information (e.g., age, weight, gender, and the like). This information may then be used to generate a classifier. The classifier, in turn, may then be used to classify the brain health status of additional test subjects.

The embodiments of system 100 illustrated in FIGS. 1A-2C may be further configured as described herein. In addition, the system 100 may be configured to perform any other step(s) of any of the method embodiment(s) described herein.

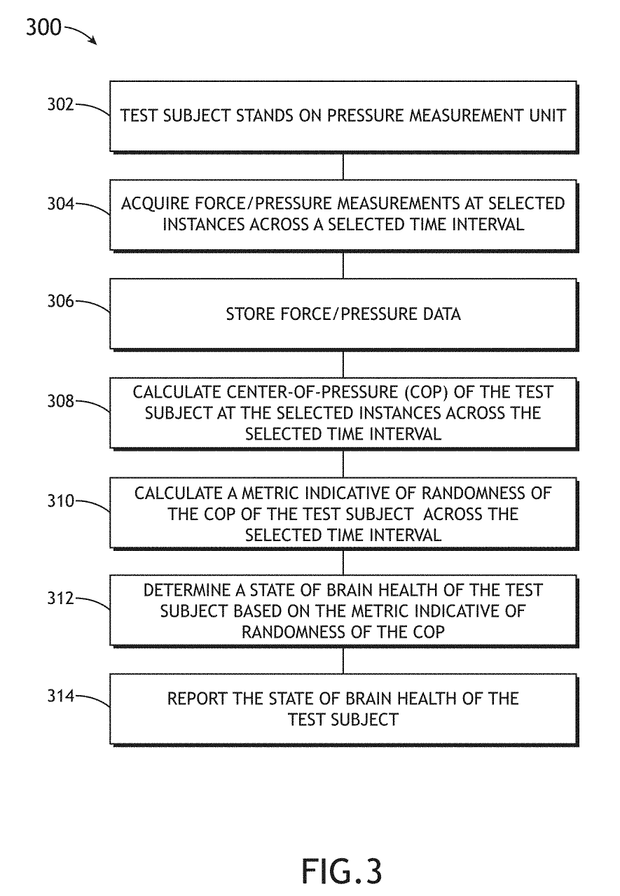

FIG. 3 illustrates a process flow diagram depicting a method 300 of identifying brain injuries, in accordance with one or more embodiments of the present disclosure. It is noted herein that the steps of method 300 may be implemented all or in part by the system 100. It is further recognized, however, that the method 300 is not limited to the system 100 in that additional or alternative system-level embodiments may carry out all or part of the steps of method 300.

In step 302, a test subject stands on a pressure measurement unit. For example, a test subject may stand on the pressure measurement unit every day for a selected time interval a suspected brain injury.

In step 304, force and/or pressure measurements are acquired at selected instances across a selected time interval. For example, as discussed previously, force sensors may be configured to transmit force/pressure measurements at one or more selected instances within a selected time interval in which a test subject stands on a pressure measurement unit. For example, if a test subject were to stand on a pressure measurement unit for one minute (e.g., time interval of one minute), force measurements may be acquired at instances separated by a hundredth of a second to ten seconds, inclusive. For instance, if a test subject were to stand on a pressure measurement unit for one minute (e.g., time interval of one minute), and force measurements were acquired at instances separated by tenths of seconds, force measurements would be acquired for six-hundred instances within the one minute time interval (10 instances/second*60 seconds=600 instances).

In step 306, force/pressure data is stored in memory. In one embodiment, in addition to storing the data, system 100 may also time stamp each data point for future reference.

In step 308, the COP of the test subject is calculated at the selected instances across the selected time interval. For example, continuing with the example above, if method 300 were configured to acquire force measurements for six-hundred instances within a one minute time interval, step 308 would calculate six-hundred COP measurements (one COP measurement for the data set acquired at each instance within the time interval).

In step 310, a metric indicative of randomness of the COP of the test subject across the selected timer interval is calculated. In one embodiment, a metric indicative of entropy may include approximate entropy.

In step 312, a state of brain health of the subject based on the metric indicative of randomness of the COP is determined. For example, the one or more processors 104 may determine the state of brain health of the test by comparing the randomness metric of the COP to one or more historical measurements of the randomness metric of the COP from the test subject. By way of another example, the one or more processors 104 may identify deteriorated brain function (e.g., occurrence of brain injury or onset of brain disease) in the test subject by identifying a decrease in the randomness metric of the test subject. By way of another example, the one or more processors 104 may identify a recovery from deteriorated brain function in the test subject by identifying an increase in randomness metric of COP of the test subject.

By way of another example, the one or more processors 104 may determine the state of brain health of the test subject based on the metric indicative of randomness of the center-of-pressure by comparing the metric indicative of randomness of the center-of-pressure to an average metric indicative of randomness of the center-of-pressure acquired from a group of individuals having one or more attributes in common with the test subject. Further, the one or more processors 104 may identify a prior brain injury by the test subject by determining the metric indicative of randomness of the center-of-pressure of the test subject is less than the average approximate entropy of the center-of-pressure acquired from the group of individuals.

In step 314, the state of the brain health of the test subject is reported. In one embodiment, reporting the test subject's brain health state may include displaying the results on a display unit of a user interface. In another embodiment, reporting the results may include transmitting an alert to a user including, but not limited to, emails, text messages, instant messages, application messages, and the like.

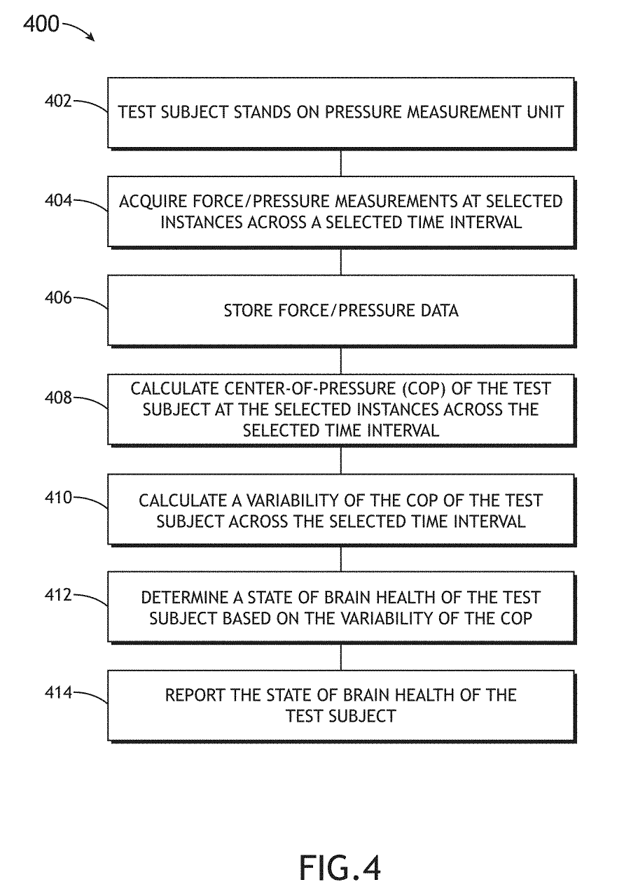

FIG. 4 illustrates a process flow diagram depicting a method 400 of identifying brain injuries, in accordance with one or more embodiments of the present disclosure. It is noted herein that the steps of method 400 may be implemented all or in part by the system 100. It is further recognized, however, that the method 400 is not limited to the system 100 in that additional or alternative system-level embodiments may carry out all or part of the steps of method 400.

In step 402, a test subject stands on a pressure measurement unit. In step 404, force and/or pressure measurements are acquired at selected instances across a selected time interval. In step 406, force/pressure data is stored in memory. In step 408, the COP of the test subject is calculated at the selected instances across the selected time interval. In step 410, a variability of the COP of the test subject across the selected timer interval is calculated. In step 412, a state of brain health of the subject based on the variability of the COP is determined. In step 414, the state of the brain health of the test subject is reported.

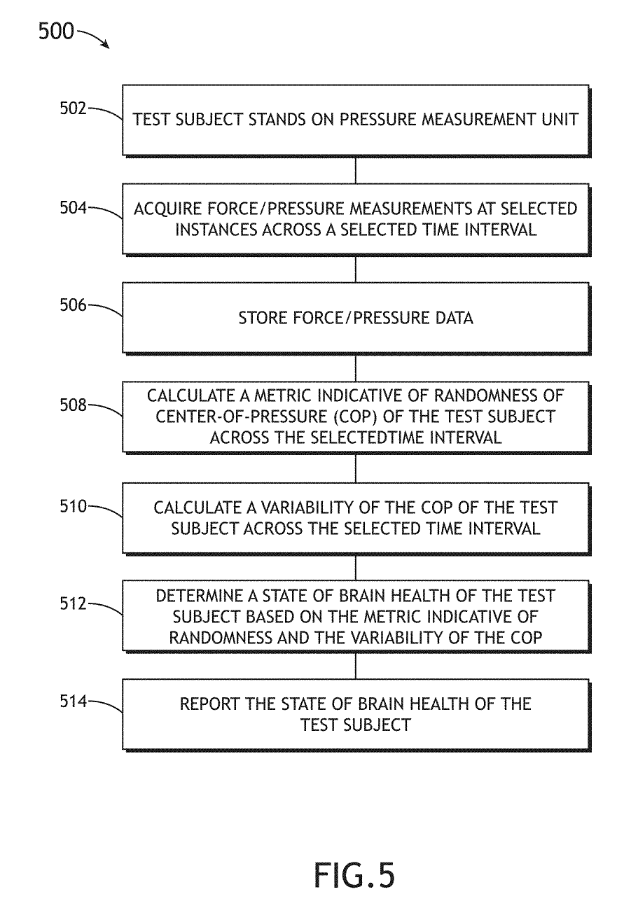

FIG. 5 illustrates a process flow diagram depicting a method 500 of identifying brain injuries, in accordance with one or more embodiments of the present disclosure. It is noted herein that the steps of method 500 may be implemented all or in part by the system 100. It is further recognized, however, that the method 500 is not limited to the system 100 in that additional or alternative system-level embodiments may carry out all or part of the steps of method 500.

In step 502, a test subject stands on a pressure measurement unit. In step 504, force and/or pressure measurements are acquired at selected instances across a selected time interval. In step 506, force/pressure data is stored in memory. In step 508, a metric indicative of randomness of the COP of the test subject across the selected time interval is calculated. In step 510, a variability of the COP of the test subject across the selected time interval is calculated. In step 512, a state of brain health of the subject based on the metric indicative of randomness and the variability of the COP is determined.

In step 514, the state of the brain health of the test subject is reported.

All of the methods described herein may include storing results of one or more steps of the method embodiments in a storage medium. The storage medium may include any storage medium described herein or any other suitable storage medium known in the art. After the results have been stored, the results can be accessed in the storage medium and used by any of the method or system embodiments described herein, formatted for display to a user, used by another software module, method, or system, and the like

It is further contemplated that each of the embodiments of the method described above may include any other step(s) of any other method(s) described herein. In addition, each of the embodiments of the method described above may be performed by any of the systems described herein.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected,” or “coupled,” to each other to achieve the desired functionality, and any two components capable of being so associated can also be viewed as being “couplable,” to each other to achieve the desired functionality. Specific examples of couplable include but are not limited to physically mateable and/or physically interacting components and/or wirelessly interactable and/or wirelessly interacting components and/or logically interacting and/or logically interactable components.

Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” and the like). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, and the like” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, and the like). In those instances where a convention analogous to “at least one of A, B, or C, and the like” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, and the like). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

1. A system for identifying brain injury in a test subject comprising:
a pressure measurement unit including a plurality of force sensors, wherein a particular force sensor is configured to output a signal indicative of a force applied to the particular sensor;
memory;
one or more processors, the one or more processors communicatively coupled to each of the force sensors, the one or more processors configured to execute a set of program instructions stored in the memory, the set of program instructions configured to cause the one or more processors to:
receive a set of signals, acquired at a selected number of instances across a selected time interval, indicative of force from at least some of the plurality of force sensors when a test subject stands on the pressure measurement unit;
calculate a center-of-pressure of the test subject at the selected number of instances across the selected time interval based on the received set of signals from the at least some of the plurality of force sensors;
calculate a metric indicative of randomness of the center-of-pressure of the test subject across the selected time interval;
determine a state of brain health of the test subject based on the metric indicative of randomness of the center-of-pressure across the selected time interval; and
report the state of brain health of the test subject to a user interface.

2. The system of claim 1, wherein the set of program instructions are configured to cause the one or more processors to determine the state of brain health of the test subject based on the metric indicative of randomness of the center-of-pressure by comparing the metric indicative of randomness of the center-of-pressure to one or more historical measurements of the metric indicative of randomness of the center-of-pressure from the test subject.

3. The system of claim 1, wherein the set of program instructions are configured to cause the one or more processors to identify deteriorated brain function in the test subject by identifying at least one of a decrease or an increase in the metric indicative of randomness of the center-of-pressure of the test subject.

4. (canceled)

5. The system of claim 1, wherein the set of program instructions are configured to cause the one or more processors to determine the state of brain health of the test subject based on the metric indicative of randomness of the center-of-pressure by comparing the metric indicative of randomness of the center-of-pressure to an average metric indicative of randomness of the center-of-pressure acquired from a group of individuals having one or more attributes in common with the test subject.

6. The system of claim 5, wherein the set of program instructions are configured to cause the one or more processors to identify a prior brain injury of the test subject by determining the metric indicative of randomness of the center-of-pressure of the test subject is less than the average approximate entropy of the center-of-pressure acquired from the group of individuals.

7. The system of claim 1, wherein the metric indicative of randomness includes approximate entropy.

8. The system of claim 1, wherein the state of brain health comprises at least one of a healthy brain function or a deteriorated brain function.

9. (canceled)

10. The system of claim 8, wherein the deteriorated brain function comprises at least one of one or more brain injuries or one or more brain diseases.

11. (canceled)

12. The system of claim 1, wherein the set of program instructions are configured to cause the one or more processors to calculate a variability of the center-of-pressure of the test subject based on the center-of-pressure of the test subject at the selected number of instances across the selected time interval.

13. The system of claim 7, wherein the set of program instructions are configured to determine a state of brain health of the test subject based on the metric indicative of randomness of the center-of-pressure across the selected time interval and the variability of the center-of-pressure of the test subject

14. The system of claim 1, wherein the pressure measurement unit comprises:
a force plate, wherein the force place includes an array of force sensors.

15. The system of claim 1, wherein the pressure measurement unit comprises:
a digital scale.

16. The system of claim 1, wherein at least some of the force sensors comprise:
at least one of one or more piezoelectric sensors, one or more force sensitive resistors (FSRs), or one or more force sensitive capacitors.

17. (canceled)

18. (canceled)

19. The system of claim 1, wherein the one or more processors and memory are included in at least one of a local controller or a remote server.

20. (canceled)

21. A system for identifying brain injury in a test subject comprising:
a pressure measurement unit including a plurality of force sensors, wherein a particular force sensor is configured to output a signal indicative of a force applied to the particular sensor;
memory;
one or more processors, the one or more processors communicatively coupled to each of the force sensors, the one or more processors configured to execute a set of program instructions stored in the memory, the set of program instructions configured to cause the one or more processors to:
receive a set of signals, acquired at a selected number of instances across a selected time interval, indicative of force from at least some of the plurality of force sensors when a test subject stands on the pressure measurement unit;
calculate a center-of-pressure of the test subject at the selected number of instances across the selected time interval based on the received set of signals from the at least some of the plurality of force sensors;
calculate a variability of the center-of-pressure of the test subject across the selected time interval;
determine a state of brain health of the test subject based on the metric indicative of randomness of the center-of-pressure across the selected time interval; and
report the state of brain health of the test subject to a user interface.

22. The system of claim 21, wherein the set of program instructions are configured to cause the one or more processors to determine the state of brain health of the test subject based on the variability of the center-of-pressure by comparing the variability of the center-of-pressure to one or more historical measurements of the variability of the center-of-pressure from the test subject.

23. The system of claim 21, wherein the set of program instructions are configured to cause the one or more processors to identify a deteriorated brain function in the test subject by identifying at least one of a decrease or increase in variability of the center-of-pressure of the test subject.

24. (canceled)

25. The system of claim 21, wherein the set of program instructions are configured to cause the one or more processors to determine the state of brain health of the test subject based on the variability of the center-of-pressure by comparing the variability of the center-of-pressure to an average variability of the center-of-pressure acquired from a group of individuals having one or more attributes in common with the test subject.

26. The system of claim 25, wherein the set of program instructions are configured to cause the one or more processors to identify a prior brain injury of the test subject by determining the variability of the center-of-pressure of the test subject is less than the average variability of the center-of-pressure acquired from the group of individuals.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A system for identifying brain injury in a test subject comprising:
a pressure measurement unit including a plurality of force sensors, wherein a particular force sensor is configured to output a signal indicative of a force applied to the particular sensor;
memory;
one or more processors, the one or more processors communicatively coupled to each of the force sensors, the one or more processors configured to execute a set of program instructions stored in the memory, the set of program instructions configured to cause the one or more processors to:
receive a set of signals, acquired at a selected number of instances across a selected time interval, indicative of force from at least some of the plurality of force sensors when a test subject stands on the pressure measurement unit;
calculate a metric indicative of randomness of a center-of-pressure of the test subject across a selected time interval;
calculate a variability of the center-of-pressure of the test subject across the selected time interval;
determine a state of brain health of the test subject based on the metric indicative of randomness of the center-of-pressure and the variability of the center-of-pressure; and
report the state of brain health of the test subject to a user interface.

39. (canceled)

40. (canceled)

41. (canceled)
